(19) United States
(12) Patent Application Publication
(10) Pub. No.: US 2007/0042064 A1
(43) Pub. Date: Feb. 22, 2007

(54) Extracts of durian fruit for use in skin care compositions

(75) Inventors: Jesse Leverett, Rockford, MI (US); Amitabh Chandra, Ada, MI (US); Jatinder Rana, Grand Rapids, MI (US); David J. Fast, Grand Rapids, MI (US); Stephen R. Missler, Grand Rapids, MI (US); David M. Flower, Caledonia, MI (US)

Correspondence Address: IN RE: ALTICOR INC. 28533;BRINKS, HOFER, GILSON & LIONE, P.O. BOX 10395, CHICAGO, IL, 60610, US

(73) Assignee: Access Business Group International LLC

(21) Appl. No.: 11/208,148

(22) Filed: Aug. 19, 2005

(51) Int. Cl. A61K 36/185

(52) U.S. Cl. 424/777

(57) ABSTRACT

The present invention relates to compositions comprising extracts of Durio zibethinus. The compositions of the present invention are useful for improving the appearance, texture, and/or moisture of mammalian skin. In particular, the compositions of the present invention decrease inflammation in the skin, inhibit matrix metalloprotease-9, which degrades skin proteins such as collagen and elastin, and inhibit melanogenesis in the skin.

### BACKGROUND

A primary goal of cosmetic science is improvement of the outward appearance and health of skin. Less desirable skin traits include wrinkles, fine lines, age spots, uneven skin tone and sagging, which all indicate aged or unhealthy skin. Thus, much of cosmetic science is targeted at treating underlying conditions that cause or stimulate the signs of skin aging.

Evidence suggests that cumulative oxidative damage, incurred throughout one's lifetime, causes skin to appear aged or unhealthy. Poor diet, lack of exercise, and exposure to ultra violet light all cause oxidative damage. Indeed, ultra violet light is known to generate reactive oxygen species (ROS), such as superoxide, singlet oxygen, hydroxyl radicals, hydrogen peroxide, and reactive nitrogen species (RNS) in the skin. These ROS/RNS are known to degrade collagen in the skin and to decrease the ability of fibroblasts to produce collagen.

Collagen is the primary protein of connective tissue, which includes cartilage, bone, tendon, teeth, and skin. Collagen (in a pre-processed form called pro-collagen) is assembled in cells and consists of three polypeptides wound around each other in a triple helix form, which is stabilized by intrachain disulfide bonds. After the helical molecule is assembled and modified in the cell it is secreted into the extracellular medium and further processed to a mature form (tropocollagen).

Matured collagen molecules assemble into fibrils in the extracellular space in a staggered, parallel, fashion wherein the molecules are stabilized in this fibril pattern by covalent cross-linking bonds between the N-terminus of one molecule and the C-terminus of another. The collagen fibrils are interlaced and branched in skin.

These interlaced, branched collagen fibrils provide the skin with its shape and firmness, while another skin protein, elastin, provides skin with its elasticity. Elastin coils and recoils like a spring and accounts for the elasticity of structures such as the skin, blood vessels, heart, lungs, intestines, tendons, and ligaments. Elastin is normally not produced by the human body after puberty and aging begins.

Like all other proteins in the human body, collagen and elastin are constantly being degraded. ROS/RNS play a role in the degradation of these skin proteins by stimulating matrix metalloprotease (MMP) enzymes. MMP enzymes degrade collagen and elastin. For example, collagenase is an MMP enzyme produced by fibroblast like synoviocytes that degrades collagen. Elastase is another MMP enzyme that degrades elastin. Another enzyme, MMP-1, cleaves fibrillar collagens, such as types I, II, and III, resulting in denatured collagens (gelatins). These denatured collagens are further degraded by MMP-9, which also degrades laminin and type IV collagen. Thus, MMP enzymes are involved in the reduction of collagen and elastin in the skin, which leads to the appearance of fine lines, wrinkles, age spots, and sagging skin.

Like loss of collagen and elastin due to oxidative damage from ROS/RNS and MMPs, loss of moisture and increased inflammatory responses contribute to skin aging. Indeed, the skin's capacity to inhibit inflammatory responses and retain water decreases with age, making the skin more vulnerable to dehydration and wrinkling. Lipids and fats in the skin help combat water loss by providing an epidermal barrier. This barrier hinders the growth of bacteria, which can cause skin irritation and sensitivity, which leads to increased inflammation and contributes to aging of skin.

In addition to lacking fine lines, wrinkles, and sagging, young, healthy skin often appears even in tone or color.

Therefore, a composition containing nutrients that inhibit MMPs and thereby help prevent the loss of collagen and elastin, nutrients that hydrate the skin, increase the synthesis of lipids in the skin, or inhibit inflammation of the skin, and nutrients that lighten or increase the evenness of skin tone would be useful for improving the appearance, texture, and moisture of the skin and for maintaining general skin health.

### BRIEF SUMMARY

Skin aging is directly related to many causes including oxidative stress of the skin, loss of moisture from the skin, and degradation of important proteins in skin such as collagen and elastin. The present invention is a formulation comprising extracts of Durio zibethinus (“durian”) and a method of using these extracts to improve the moisture, texture, and appearance of the skin. Although durian fruits are known as a food source in the pacific southeast and much of Asia, the current state of the art concerning durian extract does not recognize that durian extracts can be used to improve the appearance and health of skin. In particular, the durian extracts of the present invention have the ability to function as an anti-inflammatory. Additionally, the durian extracts of the present invention inhibit induction of MMPs and thereby decrease the degradation of important skin proteins such as collagen and elastin. Even further, the durian extracts of the present invention can be used to lighten skin tone and/or make it appear more even by inhibiting melanogenesis in mammalian skin.

The formulations of the present invention, which comprise an extract of Durian zibethinus and an acceptable carrier, are preferably topically administered. Alternatively, the formulations of the present invention may be orally administered, administered by injection, peritoneally administered, or any combination thereof.

Accordingly, in one embodiment, the present invention provides a composition comprising an extract of Durio zibethinus and an acceptable carrier, wherein the composition is effective for improving the appearance and health of mammalian skin.

Another embodiment of the invention is a method of improving the appearance and health of mammalian skin comprising topically administering a composition comprising an extract of Durio zibethinus and an acceptable carrier.

In an alternative embodiment, the invention is a method of decreasing inflammation in mammalian skin comprising administering a composition comprising an extract of Durio zibethinus and an acceptable carrier.

In a further embodiment of the invention, the invention is a method of inhibiting the induction of matrix metalloprotease-9 in mammalian skin comprising administering a composition comprising an extract of Durio zibethinus and an acceptable carrier.

In an alternative embodiment, the invention is a method of lightening or evening skin tone comprising inhibiting melanogenesis in mammalian skin by administering a composition comprising an extract of Durio zibethinus and an acceptable carrier.

### DETAILED DESCRIPTION

It is to be understood that this invention is not limited to the particular methodology or protocols described herein. Further, unless defined otherwise, all technical and scientific terms used herein have the same meaning as commonly understood by one of ordinary skill in the art to which this invention belongs. It is also to be understood that the terminology used herein is for the purpose of describing particular embodiments only, and is not intended to limit the scope of the present invention, which will be limited only by the claims.

The present invention is based on the surprising discovery that extracts of Durio zibethinus improve skin moisture, texture, and appearance. In particular, the durian extracts of the present invention have the ability to act as anti-inflammatories. The durian extracts of the present invention also prevent the degradation of important skin proteins, such as collagen and elastin, by inhibiting MMPs. Durian extracts of the present invention inhibit melanogenesis in mammalian skin and therefore, also are useful for lightening or evening skin tone.

Mammalian skin consists of two main parts: the epidermis, which is the outermost layer; and the dermis, which is collagen-rich and largely composed of connective tissues. The epidermis is the visible, defining component of the skin. The dermis generates cells, nutrients and other molecules that support and replenish the cells of the epidermis. The epidermis suffers more direct, frequent, and damaging encounters with the external world than the dermis. Thus, the epidermis is the portion of the skin that evidences visible signs of skin damage and aging, including wrinkles, fine lines, age spots, and sagging. Therefore, the durian extracts of the present invention target cells in the epidermis but also may penetrate through the epidermis to target cells in the dermis. For example, melanocytes are located both in the dermis and among the basal cells of the epidermis. Melanocytes secrete melanin, the protein which provides pigmentation to the skin. The durian extracts of the present invention inhibit melanocytes in both the epidermis and the dermis to produce steady levels of melanin resulting in a more even and/or lighter skin tone or color.

It is estimated that there are at least 28 different species of the genus Durio in Malaysia. Only five of these species, in addition to durian, are believed to bear edible fruit. These are D. dulcis, D. grandiflorus, D. graveolens, D. kutejensis, and D. oxleyanus. All Durio species are members of the Bombacaceae family. One of ordinary skill in the art will appreciate that the durian extract of the present invention may be obtained from any Durio species if it is being topically administered. One of ordinary skill in the art also will appreciate that if the durian extract of the present invention is orally administered, it preferably is obtained from an edible Durio species. Additionally, in a preferred embodiment, the durian extract of the present invention is an extract of Durio zibethinus.

Durian extracts may be made from any part of the durian plant including but not limited to the fruit, seeds, leaves, stems, bark, or roots. Durian plants generally grow to be trees reaching heights of 90 to 130 feet. Durian trees typically have a rough, peeling trunk that is approximately 4 feet in diameter. Generally, durian trees have an irregular dense or open crown of rough branches with tiny branchlets that are covered with coppery or gray scales when young.

Durian leaves are evergreen and oblong-lance-oblate, or elliptic-obovate, with a rounded base and abruptly pointed apex. Durian leaves have a leathery texture and glossy dark green or silvery, pale yellow color. They are covered with gray or reddish-brown, hairy scales on the underside.

Durian trees produce malodorous flowers that are whitish to golden-brown. The flowers typically have 3 petals and are 2 to 3 inches wide. Durian flowers produce the durian fruits, which are typically ovoid or ovoid-oblong to nearly round. Durian fruits generally are 6 to 12 inches long and 5 to 6 inches wide. They have a yellow or yellowish-green rind that is thick, tough, semi-woody, and densely set with stout, sharply pointed spines. The inner part of the durian fruit is typically divided into five compartments containing creamy-whitish, yellowish, pinkish, or orange-colored flesh, called aril, and 1 to 7 chestnut like seeds. The seeds are typically ¾ of an inch to 2¼ inches and have long, glossy, red-brown seed coats.

One of ordinary skill in the art will appreciate that the durian extract used in practicing the method of the present invention can be made using several extraction methods with different parts of the durian plant. For example, a durian extract could be made from the outer green, spiny rind, from the inner, soft rind, from the fruit pulp, or from the seeds. One example of an extraction method that can be used to produce the durian extract of the present invention is extraction with an organic solvent such as methanol, hexane, ethyl acetate, ethanol, or hydro-ethanol. For example, 4 grams of a durian material may be extracted with 25 mL of methanol.

In another example, supercritical fluid extraction techniques can be used to obtain a durian extract of the present invention. In this extraction procedure, the durian material is not exposed to any organic solvent. Rather, the extract solvent is carbon dioxide, with or without a modifier, in supercritical conditions (e.g., >31.3° C. and >73.8 bar). That is, the CO₂ is compressed by varying pressure and temperature until the CO₂ is in a liquid state. Those of skill in the art will appreciate that temperature and pressure conditions can be varied to obtain liquid CO₂ samples of differing densities. These differing CO₂ samples will display a wide-range of solvent strengths and may be used to extract the durian sample. Those of skill in the art will appreciate that one advantage of using supercritical fluid extraction techniques is that these techniques involve the use of only a single solvent, e.g., CO₂. Additionally, the single solvent, CO₂, evaporates after extraction thereby eliminating separation steps. Emulsifiers, such as anionic, cationic, amphoteric, and nonionic emulsifiers can also be used as solvents.

In a further example of an extraction technique that might be used in practicing the present invention, solvent sequential fractionation may be used to extract durian. For example, using this technique, durian can be sequentially extracted with hexane, ethyl acetate, ethanol, and hydro-ethanol. The extracts obtained after each step (fractions) of the sequence will contain chemical compounds in increasing order of polarity similar to the solvents used for extracting them. The fractions are dried to evaporate the solvents, resulting in a durian extract. Those of skill in the art will appreciate that many other solvents can be used in practicing the solvent sequential fractionation extraction of durian.

Total hydro-ethanolic extraction techniques might also be used to obtain a durian extract of the present invention. Generally, this is referred to as a lump-sum extraction of durian material. The extract generated in this process will contain all phytochemicals present in the durian material including fat and water solubles. Following collection of the extract, the solvent will be evaporated, resulting in a durian extract.

Total ethanol extraction may also be used in the present invention. This technique also uses durian material, but ethanol, rather than hydro-ethanol, is the solvent. This extraction technique generates a durian extract that may include fat soluble and/or lipophilic compounds in addition to water soluble compounds.

Those of skill in the art will appreciate that there are many other extraction processes, both known in the art and described in various patents and publications that can be used to obtain the durian extract to be used in practicing the present invention.

Methods of Administration

Improved skin appearance, texture, and moisture can be achieved by administering a formulation of the present invention externally, internally, or some combination thereof. Preferably, the formulations of the present invention are administered with an acceptable carrier. For example, the formulation of the present invention could be topically administered with an acceptable carrier in the form of a gel, lotion, cream, tonic, emulsion, etc. As a further example, the formulation of the present invention could be internally administered with an acceptable carrier in the form of a pill, tablet, powder, bar, beverage, etc. Thus, the formulations described herein are useful in a wide variety of finished products, including pharmaceutical products, food products, and beverage compositions. Preferably, the products are useful for providing mammalian skin with an improved texture, appearance, and increased moisture.

When the formulations of the present invention are orally administered in the form of a liquid, the liquid may be water-based, milk-based, tea-based, fruit juice-based, or some combination thereof. Solid and liquid formulations for internal administration according to the present invention can further comprise thickeners, including xanthum gum, carbosymethyl-cellulose, carboxyethylcellulose, hydroxyporpolcellulose, methylcellulose, microcrystalline cellulose, starches, dextrins, fermented whey, tofu, maltodextrins, polyols, including sugar alcohols (e.g., sorbitol and mannitol), carbohydrates (e.g. lactose), propylene hlycol alginate, gellan gum, guar, pectin, tragacanth gum, gum acacia, locust bean gum, gum arabic, gelatin, as well as mixtures of these thickeners. These thickeners are typically included in the formulations of the present invention at levels up to about 0.1%, depending on the particular thickener involved and the viscosity effects desired.

The solid and liquid (food and beverage) formulations of the present invention can, and typically will, contain an effective amount of one or more sweeteners, including carbohydrate sweeteners and natural and/or artificial no/low calorie sweeteners. The amount of the sweetener used in the formulations of the present invention will vary, but typically depends on the type of sweetener used and the sweetness intensity desired.

In one example of a solid or liquid formulation for oral administration, the durian extract is present in an amount from about 1 μg/ml (or μg/mg) to about 10 μg/ml (or μg/mg). In another example, the durian extract is present in an amount from about 10 μg/ml (or μg/mg) to about 100 μg/ml (or μg/mg). In a further example, the durian extract is present in an amount from about 1 μg/ml (or μg/mg) to about 100 μg/ml (or μg/mg).

In another embodiment of the invention, the formulations of the present invention are topically administered in the form of a: solution, gel, lotion, cream, ointment, oil-in-water emulsion, water-in-oil emulsion, stick, spray, paste, mousse, tonic, or other cosmetically and topically suitable form.

Preferably, formulations of the present invention that are suitable for topical administration are mixed with an acceptable carrier. An acceptable carrier may act variously as solvent, vehicle, diluent or dispersant for the constituents of the composition, and allows for the uniform application of the constituents to the surface of the skin at an appropriate dilution. The acceptable carrier may also facilitate penetration of the composition into the skin.

In one example of a formulation for topical administration, the acceptable carrier forms from about 90% to about 99.99% by weight of the total composition. In other examples, the acceptable carrier will form from about 97% to 99% by weight of the total composition. The acceptable carrier may also form from about 91% to about 98% by weight of the total composition; from about 92% to about 97% by weight of the total composition; from about 93% to about 96% by weight of the total composition; or from about 94% to about 95% by weight of the total composition. The acceptable carrier can, in the absence of other cosmetic adjuncts or additives, form the balance of the composition.

The various ingredients used in practicing the present invention may be soluble or insoluble in the acceptable carrier. If all ingredients of a formulation are soluble in the acceptable carrier, then the vehicle acts as solvent. However, if all or some ingredients of a formulation are insoluble in the acceptable carrier, then those ingredients are dispersed in the carrier by means of, for example, a suspension, emulsion, gel, cream, or paste, and the like.

Thus, it will be apparent to the skilled artisan that the range of possible acceptable carriers is very broad. For example, acceptable carriers can be emulsions, lotions, creams, or tonics. Acceptable carriers can comprise water, ethanol, butylene glycol, or other various solvents that aid in penetration of the skin. Some examples of suitable carriers are described in U.S. Pat. No. 6,184,247 and in U.S. Pat. No. 6,579,516, the entire contents of which are incorporated herein by reference.

In practicing the present invention, preferably the acceptable carrier is mixed with a durian extract of the present invention, wherein the durian extract comprises about 2% to about 5% by weight of the total composition. In other embodiments, the acceptable carrier is mixed with a durian extract of the present invention, wherein the durian extract comprises about 0.99% to about 10% by weight of the total composition; about 1% to about 9% by weight of the total composition; about 2% to about 8% by weight of the total composition; about 3% to about 7% by weight of the total composition; or about 4% to about 6% by weight of the total composition.

In general, however, acceptable carriers according to the present invention may comprise, but are not limited to comprising, any of the following examples: water; castor oil; ethylene glycol monobutyl ether; diethylene glycol monoethyl ether; corn oil; dimethyl sulfoxide; ethylene glycol; isopropanol; soybean oil; glycerin; soluble collagen; zinc oxide; titanium oxide; or Kaolin.

Acceptable carriers used in the present invention may optionally comprise one or more humectants, including but not limited to: dibutyl phthalate; soluble collagen; sorbitol; sodium 2-pyrrolidone-5-carboxylate; glycerin and derivatives thereof such as glycerin 26; sodium pca; propylene glycol; sorbitol; butylene glycol; polyols; and polyhydric alcohols. Other examples of humectants that may be used in practicing the present invention can be found in the CTFA Cosmetic Ingredient Handbook, the relevant portions of which are incorporated herein by reference.

Additionally, acceptable carriers in the present invention may optionally comprise one or more emollients including but not limited to: butane-1,3-diol; cetyl palmitate; dimethylpolysiloxane; glyceryl monoricinoleate; glyceryl monostearate; isobutyl palmitate; isocetyl stearate; isopropyl palmitate; isopropyl stearate; butyl stearate; isopropyl laurate; hexyl laurate; decyl oleate; isopropyl myristate; lauryl lactate; octadecan-2-ol; caprylic triglyceride; capric triglyceride; polyethylene glycol; propane-1,2-diol; triethylene glycol; sesame oil; coconut oil; safflower oil; isoamyl laurate; nonoxynol-9; panthenol; hydrogenated vegetable oil; tocopheryl acetate; tocopheryl linoleate; allantoin; propylene glycol; arachis oil; castor oil; isostearic acid; palmitic acid; isopropyl linoleate; lauryl lactate; myristyl lactate; decyl oleate; or myristyl myristate. Other examples of emollients that may be used in practicing the present invention can be found in the CTFA Cosmetic Ingredient Handbook, the relevant portions of which are incorporated herein by reference.

Acceptable carriers used in the present invention also may optionally comprise one or more penetration enhancers including but not limited to: pyrrolidones, for example 2-pyrrolidone; alcohols, such as ethanol; alkanols, such as decanol; glycols, such as propylene glycol, dipropylene glycol, butylene glycol; glycol ethers such as dimethyl isosorbide, ethoxy diglycol; emulsifiers; or terpenes.

Other acceptable carriers that may be used in practicing the present invention will be apparent to those of skill in the art and are included within the scope of the present invention.

For example, an acceptable carrier can be a lotion that is topically applied. The lotion may comprise one or more of the following ingredients: cabomer 981, water, glycerin, isopropyl myristate, mineral oil, shea butter, stearic acid, glycol stearate, cetyl alcohol, dimethicone, preservatives, tea, and various durian extracts of the present invention.

The compositions of the present invention may also contain various known and conventional cosmetic adjuvants so long as they do not detrimentally affect the desired skin improvement and moisturizing effects provided by the formulation. For example, a composition of the present invention can further include one or more additives or other optional ingredients well known in the art, which can include but are not limited to fillers (e.g., solid, semi-solid, liquid, etc.); carriers; diluents; thickening agents; gelling agents; vitamins, retinoids, and retinols (e.g., vitamin B₃, vitamin A, etc.); pigments; fragrances; sunscreens and sunblocks; anti-oxidants and radical scavengers; organic hydroxy acids; exfoliants; skin conditioners; moisturizers; ceramides, pseudoceramides, phospholipids, sphingolipids, cholesterol, glucosamine, pharmaceutically acceptable penetrating agents (e.g., n-decylmethyl sulfoxide, lecithin organogels, tyrosine, lysine, etc.); preservatives; antimicrobial agents; amino acids such as proline, pyrrolidone carboxylic acid, its derivatives and salts, saccharide isomerate, panthenol, buffers together with a base such as triethanolamine or sodium hydroxide; waxes, such as beeswax, ozokerite wax, paraffin wax; plant extracts, such as Aloe Vera, cornflower, witch hazel, elderflower, or cucumber and combinations thereof. Other suitable additives and/or adjuncts are described in U.S. Pat. No. 6,184,247, the entire contents of which are incorporated herein by reference.

The composition of the present invention can include additional inactive ingredients, including, but not limited to emulsifiers, co-solvents, and excipients. Emulsifiers, such as hydrophilic and hydrophobic emulsifiers, can be included in the formulations. Particular emulsifiers can be used based on the overall composition and the intended delivery of the composition. Useful emulsifiers include polyethoxylated fatty acids, Polyethylene glycol (PEG)-fatty acid diesters, PEG-fatty acid mono- and diester mixtures, polyethylene glycol or glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic emulsifiers, and mixtures thereof.

The compositions of the present invention also can include co-solvents such as alcohols and polyols, polyethylene glycols ethers, amides, esters, other suitable co-solvents, and mixtures thereof. The formulations can also include excipients or additives such as sweeteners, flavorants, colorants, antioxidants, preservatives, chelating agents, viscomodulators, tonicifiers, odorants, opacifiers, suspending agents, binders, and mixtures thereof.

Generally, the compositions of the present invention are topically or orally administered at least on a daily basis for a period of time sufficient to bring about the desired level of improvement in skin appearance, texture, and moisture. Topical or oral administration of the compositions of the invention may continue for any suitable period of time. More specifically, within a few hours to within a few days of the initial administration, a user may notice the skin has an improved appearance, texture, and moisture. It should be appreciated that the frequency with which the compositions of the present invention should be applied or ingested will vary depending on the desired level of improved appearance, texture, and moisture. In particular, the degree of cosmetic enhancement will vary directly with the total amount of composition used and with the frequency of application.

Useful dosage forms can be prepared by methods and techniques that will be well understood by those of skill in the art and may include the use of additional ingredients in producing tablets, capsules, or liquid dosage forms.

It is intended that the foregoing detailed description be regarded as illustrative rather than limiting. The present invention is further illustrated by the following experimental investigations and examples, which should not be construed as limiting. The contents of all references, patents and published applications cited throughout this patent are hereby incorporated by reference herein.

### EXAMPLES

### Example 1

### Durian Extract Samples

Durian extracts to be used in the present invention may be obtained by dividing a fruit of Durio zibethinus into four portions: the outer green, spiny fruit rind; the inner, soft fruit rind; the fruit pulp; and the seeds. Each portion is sliced into small pieces, mashed, ground, and/or pulverized using standard techniques. Four grams (4 g) of each portion is then collected in separate containers. Each sample is then extracted with 25 mL of methanol. This technique produced 0.40 g of outer green, spiny, fruit rind extract (DE1), 0.50 g of inner soft fruit rind extract (DE2), 0.99 g of fruit pulp extract (DE3), and 0.46 g of seed extract (DE4).

The effect of each of each of these extracts, D1-D4, was tested in the in vitro bioassays described below. These bioassays were designed to elucidate the activity of the extract being tested within the skin.

### Example 2

### Inhibition of MMP-9 Induction Activity by Durian Extracts

MMPs are enzymes responsible for degrading collagen and elastin in the skin. For example, MMP-1 cleaves fibrillar collagens, such as types I, II, and III, resulting in denatured collagens (gelatins) that are further degraded by MMP-9, which degrades laminin and type IV collagen.

HS27 (fibroblasts) and HEK001 (keratinocytes) were co-cultured on plates. Cell cultures were pre-treated with durian extracts at concentrations of 1 μg/ml, 10 μg/ml, and 100 μg/ml. Following pre-treatment, cell cultures were treated with tumor necrosis factor alpha (TNF-α) (100 ng/ml) and transforming growth factor beta (TGF-β) (10 ng/ml) to induce MMP expression. Following treatment, cells were lysed and centrifuged. Cell supernatants were collected and tested for MMP-9 expression and activity using R&D Systems' Fluorokine assay kits (fluorescence immunoassay). These assay kits use MMP-9 specific antibodies to capture the enzyme and a fluorogenic substrate, which is cleaved by the active enzyme, to yield a fluorescence signal proportional to the amount of MMP enzyme in the sample. Active and activatable enzyme can be determined by selective use of p-aminophenylmercuric acetate (“APMA,” available from Sigma) to activate pro-MMP forms.

The results of this experiment are reported below in Table 1. “DE” stands for “durian extract.” Durian extract 1 (DE1) was obtained from the outer green, spiny rind of a durian fruit. Durian extract 3 (DE3) was obtained from the soft fleshy pulp or arial of a durian fruit. Durian extract 4 (DE4) was obtained from seeds of the durian fruit. “RFU” stands for relative fluorescent units. A higher RFU indicates more MMP activity. Results were adjusted for the dilution factor (1:50) of the samples. These results demonstrate that DE3 (at 100 μg/ml) and DE4 (at 10 μg/ml) are the most potent inhibitors of TNF-α/TGF-β stimulated induction of MMP-9.

TABLE 1
Screening Results for anti-MMP 9

| | | TNF/TGF - 50/5 μg/ml with APMA | | | |
|---|---|---|---|---|---|
| | | 1:50 DILUTION | | | |
| | | RFU | MMP-9 | Adjusted | % contr |
| DE1 | 1 μg/ml | 242.5 | 1.53 | 76.50 | 80.1% |
| DE1 | 10 μg/ml | 303 | 2.39 | 119.45 | 125% |
| DE1 | 100 μg/ml | 231.5 | 1.37 | 68.70 | 71.9% |
| DE3 | 1 μg/ml | 273 | 1.96 | 98.15 | 102.7% |
| DE3 | 10 μg/ml | 242 | 1.52 | 76.15 | 79.7% |
| DE3 | 100 μg/ml | 187 | 0.74 | 37.10 | 38.8% |
| DE4 | 1 μg/ml | 248.5 | 1.62 | 80.80 | 84.6% |
| DE4 | 10 μg/ml | 182.5 | 0.68 | 33.95 | 35.5% |
| DE4 | 100 μg/ml | 228 | 1.32 | 66.23 | 69.3% |

| | | | | |
|---|---|---|---|---|
| Control (TNF + | 269.33 | 1.91 | 95.55 | 100% |
| TFG) | | | | |

### Example 3

### Melanogenesis Inhibition

The various hues and degrees of pigmentation found in the skin and hair of mammals are directly related to the amount of melanin present in the skin or hair. Melanin is a pigment produced by melanocytes, which are cells found among the basal cells of the epidermis. The synthesis of melanin, melanogenesis, is catalyzed by the enzyme tyrosinase, which is expressed preferentially in melanocytes. Higher levels of melanin in the skin and hair of a mammal correlate to darker hair and skin color. Therefore, by inhibiting melanogenesis, it is possible to whiten or lighten skin or hair color (tone).

MakTek's MelanoDerm™ System consists of normal, human-derived keratinocytes (NHEK) and melanocytes (NHM) which have been cultured to form a multilayered, highly differentiated model of the human epidermis. The NHM cells within the co-cultures undergo spontaneous melanogenesis, which leads to tissues of varying levels of pigmentation. These tissues are produced using serum free medium without artificial stimulators of melanogenesis such as TPA and IBMX. The cultures are grown on cell culture inserts at the air-liquid interface, allowing for topical administration of test agents such as skin lighteners or self-tanning agents.

In the present example, sample durian extract obtained from each of the pulp, seeds, rind, and spiny rind were topically administered to the cells present in the MakTek MelanoDerm™ System. In particular, the durian extract samples were applied at concentrations of 1 μg/ml (or μg/mg), 10 μg/ml (or μg/mg), and 100 μg/ml (or μg/mg). The averaged results of this assay (the Melanogenesis Inhibition Assay) using durian extracts of the present invention are reported below in Table 3.

TABLE 3
Melanogenesis Inhibition Screening Results

| Sample durian | |
|---|---|
| exact obtained | % melanin |
| from: | control |
| Pulp | 77 |
| Seeds | 55 |
| Rind | 80 |
| Spiny Rind | 85 |

These results indicate the percent of control melanin from untreated control cells. Therefore, a number lower than 100 indicates inhibition of melanin synthesis. As evidenced by these results, each of the durian extracts tested has the ability to inhibit melanogenesis while the durian seed extract showed the highest levels of melanogenesis inhibition.

### Example 4

### Anti-Inflammatory Activity

Skin irritation and sensitivity lead to increased inflammation, which in turn contributes to aging of skin. Inflammation is mediated by many cellular factors including prostaglandin E2 (PGE2), granulocyte/macrophage-colony stimulating factor (GM-CSF), and interleukin-1β (IL-1β). As a positive control, PGE2 and GM-CSF cytokine activity were monitored by eliciting fibroblast/keratinocyte co-cultures with phorbol myristic acetate (PMA). Also as a positive control, IL-1β activity was monitored by eliciting fibroblast/keratinocyte co-cultures with lipopolysaccharide from THP-1 monocytes. One of ordinary skill in the art will appreciate that many methods may be used to determine what level of inflammation mediator is secreted in cell culture in response to elicitation of the cells with various compounds.

To determine the ability of a durian extract to inhibit inflammation, fibroblast/keratinocyte co-cultures also were exposed to durian extracts D1-D4 at concentrations of 10 μg/ml, and 100 μg/ml. Additionally, hydrophilic and lipophilic extracts obtained from both the whole fruit and the seeds were tested for anti-inflammatory activity. The results of this anti-inflammatory bioassay are indicated below in Table 4

TABLE 4
Anti-Inflammatory Assay Screening Results

| | PGE-2 | GM-CSF | IL-1 |
|---|---|---|---|
| | (% untreated control | (% untreated control | (% untreated control |
| Sample | n = 3, 10 μg/ml) | n = 3, 100 μg/ml) | n = 1, 10 μg/ml) |
| Pulp | 82.5 ± 51.9 | 102.1 ± 20.8 | 65.9 |
| Seeds | 96.1 ± 36.2 | 142.3 ± 31.4 | 80.2 |
| Rind | 81.4 ± 44.5 | 89.3 ± 11.2 | 153.7 |
| Spiny | 89.9 ± 44.8 | 70.6 ± 16.1 | 85.9 |
| Rind | | | |

These results indicate the expression level of an inflammation mediator (PGE-2, GM-CSF, and IL-1β) compared to untreated control cells (100%). Therefore, a number lower than 100 indicates an anti-inflammatory effect. None of the extracts inhibited secretion of all three mediators. The pulp extract was the strongest inhibitor of IL-1 while the rind extract was the strongest inhibitor of PGE-2. Similarly, the spiny outer rind extract was the most potent inhibitor of GM-CSF secretion. In a preferred embodiment, a durian extract of the present invention comprises extracts of the durian pulp and spiny rind, as these extracts inhibited secretion of one or more of the mediators, but did not augment the secretion of any mediator.

1. A composition comprising a methanol extract of durian (Durio zibethinus) and an acceptable carrier, wherein the composition is effective for one or more of the following: decreasing inflammation in skin cells, inhibiting induction of matrix metalloprotease-9 in skin cells, or inhibiting melanogenesis in skin cells.

2. The composition of claim 1, wherein the methanol extract of durian (Durio zibethinus) is present in an amount from about 0.99% to about 10% by weight of the total composition.

3. The composition of claim 2, wherein the methanol extract of durian (Durio zibethinus) is present in an amount from about 1% to about 9% by weight of the total composition.

4. The composition of claim 2, wherein the methanol extract of durian (Durio zibethinus) is present in an amount from about 2% to about 8% by weight of the total composition.

5. The composition of claim 2, wherein the methanol extract of durian (Durio zibethinus) is present in an amount from about 3% to about 7% by weight of the total composition.

6. The composition of claim 2, wherein the methanol extract of durian (Durio zibethinus) is present in an amount from about 2% to about 5% by weight of the total composition.

7. The composition of claim 1, further comprising a penetration enhancer that enhances the penetration of the methanol extract of durian (Durio zibethinus) into the epidermal and dermal cells of skin.

8. The composition of claim 7, wherein the penetration enhancer is a glycol, alcohol, or emulsifier.

9. The composition of claim 1, further comprising at least one cosmetic adjuvant comprising: solid, semi-solid, or liquid fillers; thickening agents; gelling agents; reducing agents; vitamins; retinoids; retinols; anti-oxidants; pigments; fragrances; sunscreens; sunblocks; organic hydroxy acids; exfoliants; skin conditioners; moisturizers; ceramides; phospholipids; cholesterol; hyaluronic acids; glucosamine; penetration enhancers; preservatives' antimicrobial agents; waxes; plant extracts of combinations thereof.

10. The composition of claim 1, wherein the composition decreases inflammation in the skin cells.

11. The composition of claim 1, wherein the composition inhibits induction of matrix metalloprotease-9 in the skin cells.

12. The composition of claim 1, wherein the composition inhibits melanogenesis in the skin cells.

13. A method of improving the appearance and health of a subject's skin comprising administering the composition of claim 1 to the subject.

14. The method of claim 12, wherein the composition is topically administered in the form of a gel, lotion, cream, ointment, emulsion, paste, or mousse.

15. The method of claim 13, wherein the composition is orally administered in the form of a liquid, tablet, pill, lozenge, powder, or food.

16. A method of decreasing inflammation in mammalian skin comprising administering the composition of claim 1.

17. The method of claim 16, wherein the composition is topically administered in the form of a gel, lotion, cream, ointment, emulsion, paste, or mousse.

18. The method of claim 17, wherein the composition is orally administered in the form of a liquid, tablet, pill, lozenge, powder, or food.

19. A method of inhibiting induction of matrix metalloprotease-9 in mammalian skin comprising administering the composition of claim 1.

20. The method of claim 19, wherein the composition is topically administered in the form of a gel, lotion, cream, ointment, emulsion, paste, or mousse.

21. The method of claim 19, wherein the composition is orally administered in the form of a liquid, tablet, pill, lozenge, powder, or food.

22. A method of inhibiting melanogenesis comprising administering the composition of claim 1.

23. The method of claim 22, wherein the composition is topically administered in the form of a gel, lotion, cream, ointment, emulsion, paste, or mousse.

24. The method of claim 22, wherein the composition is orally administered in the form of a liquid, tablet, pill, lozenge, powder, or food.
